(19) United States
(12) Patent Application Publication
(10) Pub. No.: US 2007/0065511 A1
(43) Pub. Date: Mar. 22, 2007

(54) Controlled delivery creatine formulations and method of using the same

(76) Inventors: Mark J. Tallon, London (GB); Robert Child, London (GB)

Correspondence Address: WELSH & KATZ, LTD, 120 S RIVERSIDE PLAZA, 22ND FLOOR, CHICAGO, IL, 60606, US

(21) Appl. No.: 11/519,297

(22) Filed: Sep. 12, 2006

(30) Foreign Application Priority Data
Dec. 9, 2004 (GB) GB 0518579.8

(51) Int. Cl. A61K 36/82; A61K 36/67; A61K 31/385; A61K 9/22; A61K 31/205; A61K 31/198; A61K 9/48; A61K 33/00

(52) U.S. Cl. 424/468; 424/722; 424/729; 424/734; 514/554; 514/440; 514/561; 424/451; 514/649

(57) ABSTRACT

Controlled delivery formulations of creatine and a method of using the same are disclosed. Creatine is combined with an encapsulation/delivery agent and/or excipient, which act in such a way as to provide controlled delivery of creatine. This delivery profile substantially increases the period of time over which levels of creatine are elevated in blood relative to conventional creatine formulations. This makes it possible to control the creatine delivered from the digestive tract to provide 0.2 to 1.5 g of creatine per hour. These features make it possible to retain a higher proportion of ingested creatine within the body tissues, including muscle. This allows controlled delivery creatine to 1) maintain muscle and other tissue creatine levels with a smaller creatine dose than provided by conventional loading and maintenance protocols and 2) produce greater elevations in body creatine stores with a smaller creatine dose.

### FIELD OF THE INVENTION

This invention relates to controlled delivery oral compositions for increasing creatine retention in the human or animal body and to a method of using the same.

### BACKGROUND OF THE INVENTION

Creatine plays a pivotal role in the regulation and homeostasis of skeletal muscle energy metabolism and it is now generally accepted that phospho/phosphoryl-creatine availability is important for the initiation and maintenance of muscle force production during intense contractions. Creatine may also be involved in other processes concerned with protein synthesis and hypertrophy of muscle fibres during training. Although creatine synthesis occurs in the liver, kidney and pancreas, it has been known for sometime that the oral ingestion of creatine will add to the whole body creatine pool. It has been shown that the ingestion of 20 to 30 g creatine monohydrate (Cr.H.sub.2 O) per day for several days can lead to a greater than 20% increase in human skeletal muscle total creatine content. Thus, U.S. Pat. No. 5,767,159 (HULTMAN) discloses the administration of creatine monohydrate in amounts of at least 15 g (or 0.2-0.4 g/kg body weight) per day, for at least 2 days, for increasing muscular strength.

It was subsequently found that 5 to 7 days supplementation with creatine monohydrate at 20 g per day results in an initial elevation of the tissue stores (creatine loading); thereafter it requires 2 to 3 g per day for a 75 kg man or woman to maintain elevated muscle creatine concentrations. Supplementation with any bioavailable source of creatine (i.e. creatine supplementation) at an appropriate dose can provide performance improvements to athletes involved in explosive events, which include all events lasting from a few seconds to a few minutes (such as sprinting, swimming, weight-lifting etc). Endurance performance in events lasting longer than about 30 minutes appear less affected by creatine supplementation, except where this involves short periods when the energy demand exceeds the phosphocreatine (PCr) threshold (Sahlin 1990); particularly when the local muscle carbohydrate stores have become depleted. Creatine is not a drug but a normal component of foods, particularly meats and fish. It has been shown however over a period of several months following initial creatine loading that the muscle creatine concentration gradually falls when providing a maintenance dose of 2 to 5 g per day (Derave et al. 2003).

Contemporary thinking by leading creatine experts (Derave et al. 2003, Mesa et al. 2002, Bemben and Lamont 2005 Sports Medicine 35: 107-125) has centred on 2 mechanisms of increasing creatine retention. The first concept is to co-ingest creatine and carbohydrates (as disclosed in U.S. Pat. No. 5,968,900 Greenhaff) and the second is to provide creatine “intermittently or cycled”, i.e. supplementation for 2 to 6 weeks followed by 2 weeks without supplementation, thus teaching the need to generate high plasma creatine concentrations.

Comparable to U.S. Pat. No. 5,968,900 (Greenhaff), an additional disclosure of a proposed method of increasing creatine transport can be found in US 2003/0215506 (KUHRTS) which discloses co-ingestion of creatine with IGF-1. Additionally, WO/01/35953 (KUHRTS) discloses formulations of creatine in combination with an insulin-modulating agent in an attempt to increase creatine transport. These applications make claims for increased creatine transport, which relates to the movement of compounds from one place to another, while the novel formulations of the claimed invention increase creatine retention within the body. Although creatine transport and creatine retention are related, those experienced in the art will recognise that the claim of increased creatine retention means that creatine is maintained within tissues while claims of increased transport, or transport rate, do not necessitate that the creatine is actually retained within body tissues.

Thus, in the above conventional non-controlled creatine formulations, the creatine is liberated quickly, and a large proportion rapidly enters the circulatory system and is metabolised in the liver and/or excreted in the urine. Ultimately, this reduces the duration that plasma creatine is maintained at Vmax (+/−30%), thereby reducing muscle creatine uptake and therefore creatine retention.

In contrast, the novel formulations of the present invention precisely control the release of creatine to provide a specific concentration of creatine in plasma, allowing this optimal plasma concentration to be maintained for a longer time period. Elevation of muscle creatine is achieved with the novel formulation of the present invention without the need for high plasma creatine concentrations and without the need to co-ingest creatine with other compounds, which until now have been considered necessary by those experienced in the art to increase creatine transport and retention.

The novel formulations of the present invention thus provide important advantages over known formulations. For example, the claimed formulations do not rely on insulin-mediated transport; nor do they require additional bioactives in order to increase creatine transport and retention. Therefore, the total amount of material to be ingested with creatine is significantly less. This provides increased formulation flexibility for product manufacturers as the claimed creatine formulations provide a physiologically efficacious creatine dose in capsule or tablet form. Furthermore, the absence of nutritional bioactives to increase creatine transport also negates the requirement to consume substantial amounts of calories and results in cost savings.

It has been found that creatine stimulates satellite cell (SC) proliferation and differentiation in cell cultures (Vierck et al., 2003). Furthermore, findings in rats showed that creatine supplementation in combination with an increased functional load by synergist ablation induced increased satellite cell mitotic activity (Dangott et al., 2000), demonstrating that creatine may play a role in the activation of SC in muscle undergoing hypertrophy. Recent work has investigated the influence of creatine, protein and carbohydrate supplementation on satellite cell (SC) frequency and myonuclear number in human skeletal muscle with resistance training (Olsen 2004). Total muscle creatine (TCr) was elevated compared to baseline at weeks 4 and 8 during the supplementation and training program (Olsen 2004). During the following 8 weeks TCr decreased, and at week 16 it was not significantly different from pre-supplementation. SC/fibre showed a significant time effect from baseline in CRE at week 4 (111%) and week 8 (93%), however at week 16 no difference could be observed compared to baseline (Olsen 2004). This evidence suggests new SC fibre number is directly related to the maintenance of high muscle creatine levels during resistance exercise, which cannot be maintained beyond 8 weeks, as described in the work above and in other studies (Derave et al. 2004; Eijnde et al. 2003; Vandenberghe et al. 1997; Van Loon et al. 2003).

We have now unexpectedly found that by the use of controlled delivery creatine formulations, it is possible to obtain an optimal plasma profile of creatine to maximise creatine retention. This will result in a better retention of muscle creatine levels than can be achieved with conventional creatine formulations, for which delivery is uncontrolled. Furthermore, the increase in creatine retention provides more effective gains in muscle mass and strength during resistance training. Alternatively increased creatine retention would also allow greater maintenance in improvements in high intensity exercise performance and cognitive function.

### SUMMARY OF THE INVENTION

The present invention overcomes the disadvantages of conventional rapidly liberated creatine formulations. Thus, in accordance with one aspect of the invention, there is provided a controlled delivery formulation for increasing creatine retention in the human or animal body, comprising creatine or a derivative as the active ingredient and an encapsulation and/or other agent for controlling the delivery of the active ingredient whereby in use, creatine is liberated in a manner such as to increase the duration over which the plasma concentration of creatine is maintained at Vmax (+/30%) of the muscle creatine transporters, thereby causing an increase in muscle creatine uptake and its subsequent retention.

In accordance with another aspect of the invention there is provided a method other than therapeutic, surgery or diagnostics, for increasing creatine retention in the human or animal body by at least 10% as compared to an identical amount of creatine given in a conventional non-controlled formulation, the method comprising administering the controlled delivery formulation of the invention.

### Definitions

The term “creatine” is used herein to encompass all creatine analogues and associated derivatives, such as creatine monohydrate, phosphocreatine, and other salts, conjugates and chelates of creatine. Presently preferred forms of creatine are creatine citrate, creatine pyruvate and creatine ester, and chelates and conjugates thereof.

The terms “conventional creatine” and “conventional creatine formulations” are used herein to refer to creatine formulations, which have the characteristics of comparatively fast delivery and absorption profiles, relative to controlled delivery creatine formulations. Such a formulation may be in the form of a tablet, capsule, food or the like designed to provide for substantially immediate liberation of the active ingredient and includes enteric coated and/or pH sensitive oral formulations which provide some initial protection to the active ingredient and thereafter allows an immediate and substantial liberation of all the active ingredient/s.

The term “controlled delivery” for the purposes of the present invention can include 1) the delivery of the creatine bolus to the gastro intestinal tract; 2) the delivery of creatine at specific rates, as to provide a specific plasma creatine concentration range; and 3) the delivery of creatine to maximise the duration that plasma creatine is maintained within a specific concentration range.

The term “controlled delivery” is used herein as including any system where the delivery rate or time of delivery is increased relative to conventional creatine. By definition (for a creatine bolus in the range 2 to 20 g) this will reduce the maximum creatine concentration (Cmax in FIG. 2) and will increase the time that the plasma concentration is maintained at Vmax +/−30% (see FIG. 2). This invention is concerned with both individual and combined aspects 1, 2, and 3 of controlled delivery creatine systems.

The term “serum” is used herein as referring to the fluid portion of blood prepared without the use of anticoagulants and the term “plasma” refers to the fluid portion of blood prepared using anticoagulant. The term blood refers to both the fluid portion and cells naturally present in blood. This invention is concerned with delivery of creatine to muscle tissues via blood and encompasses both serum and plasma, hence the terms plasma, serum and blood can be used interchangeably.

The term “delivery/encapsulation agent” is used herein as meaning any compound, or physical or chemical system, forming a part of the formulation, which acts to provide a controlled delivery of the creatine, including organic or synthetic ingredients whose effect is to slow or delay the delivery of creatine by more than 10% as compared to conventional creatine.

The term “excipient material” refers to any compound other than the active ingredient, which acts in a known manner as a carrier or diluent or the like, whether or not these provide any nutritional benefits as an adjunct. Examples of excipient materials are an orally ingested active consisting of inhibitors of liver creatine disposal such as black pepper (or black pepper extracts such as bioperine); or/and sodium; or/and potassium; or/and synephrine; or/and 4-hydroxyisoleucine; or/and green tea or its actives; or/and lipoic acid or/and camosine.

The term “chemical degradation” is intended to mean herein that the active ingredient (creatine) is subjected to chemical reactions, which reduce or prevent its ability to increase creatine or phosphocreatine levels in body tissues including muscle.

### BRIEF DESCRIPTION OF DRAWINGS

Reference will now be made to the accompanying drawings.

FIG. 1 Pharmacokinetic evaluation of changes in plasma creatine concentrations after consumption of a 5 g bolus of conventional creatine in humans.

FIG. 1 illustrates the changes in plasma creatine concentrations following ingestion of a 5 g bolus of conventional creatine in humans. Cmax is the maximum creatine concentration in plasma; Vmax is the plasma creatine concentration that results in the maximum rate of muscle creatine uptake and Km refers to Michaelis constant of the muscle creatine transporters. Following extensive evaluation of creatine pharmacokinetics in humans and development of mathematical modelling techniques exclusive to CR-technologies, we describe the optimal creatine delivery profile that results in maximum creatine retention within the body. This creatine profile can be applied to a single creatine bolus in the range 2 to 20 g.

FIG. 2 Conceptualised graph comparing the change in plasma creatine concentrations following ingestion of an oral 5 g creatine bolus of either conventional creatine or controlled delivery creatine.

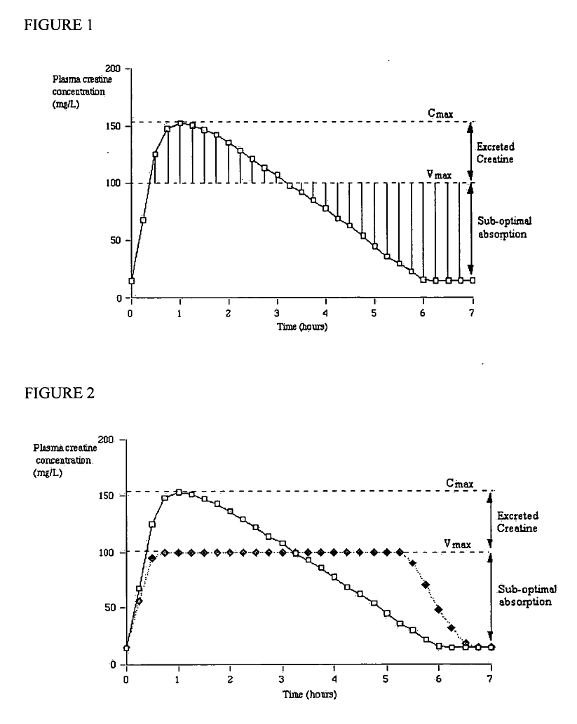

The open square with the unbroken dark line (-□-) represents conventional creatine (which is liberated quickly) and the shaded diamond with the light dashed line (-♦-) represents the controlled delivery creatine. Note that Cmax is lower for the controlled delivery creatine, resulting in less creatine degradation by the liver, urinary loss and renal load. It is also important to note that the time at Vmax is almost double that of conventional creatine, resulting in greater muscle creatine uptake.

### DETAILED DESCRIPTION

In accordance with the invention it is possible to allow creatine to be maintained at a specific plasma concentration that results in maximal rates of muscle creatine uptake for a longer time, causing a substantial increase in muscle creatine uptake.

The formulations of the invention are preferably to be delivered as an oral dosage formulation such as, for example, tablets, capsules, caplets, suspensions, and solid food forms. These include formulations presented in liquid media including gel preparations and aqueous solutions (such as drinks), or in which creatine is suspended in, or surrounded by, a matrix material such as a solid food including but not exclusive to chewing gum or hard sweet or tablet or capsule. The creatine may be presented in a combination of these matrices, such as a tablet, or sweet, or gum, with a gel or liquid or powder or gum centre.

The dosage may be of any desired size in terms of the creatine ingredient. Generally from 250 to 5,000 mg of the creatine ingredient dosage unit.

A typical formulation contains from about 50% to 99% by weight of creatine. A preferred formulation will comprise 50-70% by weight of creatine active ingredient with the remainder being the delivery/encapsulation agent and/or excipient, which slows and/or delays creatine delivery by more than 10% relative to conventional creatine formulations. More preferably, the formulation comprises 55% to 65% of active ingredient and even more preferably about 60% of active ingredient by weight. Thus, a particularly preferred oral formulation of the invention comprises a ratio of creatine to delivery agent and/or excipient in the ratio of 3:2 by mass. Each individual dosing will provide between 2 and 5.0 g of creatine, with up to four individual doses per day. Hence the daily creatine dose is in the range 2 to 20 g daily.

The formulation is characterized by (a) protecting the active ingredient from chemical degradation in the gastrointestinal tract and (b) delivering the active ingredient in a controlled manner i.e. at a rate of 0.5 to 1.5 g per hour. By gradually releasing the active ingredient the plasma levels of creatine obtained are (1) lower than those obtained with single dose non-controlled delivery formulation; and (2) maintained over longer periods of time than obtained with single dose non-controlled delivery formulation and (3) urinary creatine excretion is decreased, demonstrating greater retention of creatine within body tissues including but not constrained to muscle. Specifically the invention liberates the active ingredient so as to obtain a serum or plasma creatine concentration in humans of 100 mg/l, or close to 100 mg/l of plasma, +/−30%. Hence the acceptable plasma/serum creatine concentration range is from 70 mg/l to 130 mg/l.

Some characteristics of creatine are (1) it is non-toxic at relatively high levels. Hence the level of creatine intake required to enhance muscle performance, is many times lower than that needed to produce toxic effects in healthy humans and (2) creatine is quickly absorbed in the human gut and transported into skeletal muscle tissues. The present invention relies in part on the discovery that creatine provides desirable results even at very low levels of intake provided those low intake levels are maintained over an extended period of time as achieved with the creatine formulation described herein.

The creatine plasma level obtained using conventional creatine delivery (i.e. non-controlled delivery) systems is insufficient to maintain maximal creatine stores measured following creatine “loading”. Conventional creatine fails to maintain plasma creatine levels at concentrations where urinary creatine losses are minimized; or maintain the plasma creatine concentration at a sufficiently high level to ensure that muscle creatine transporters are saturated for a sufficient duration that creatine uptake is maximised. By providing a prolonged elevation of creatine at a level lower than that obtained with conventional creatine of 2 to 5 g, whole body creatine retention can be improved, leading to higher creatine stores.

The methods, (other than therapeutic, surgery or diagnostic) and controlled delivery formulations of the invention may be used to increase bodily creatine retention in humans. The ability to increase creatine retention may also be desired in individuals having relatively low general creatine levels, for example vegetarians and/or vegans who do not consume meat, and sufferers of diseases and genetic myopathies/cytopathies that affect muscle. Other body tissues may also benefit from greater creatine uptake, including those of brain and/or spinal cord. A further example where the invention may be applied is to help in the maintenance of optimal creatine levels in the brain and/or spinal cord during sleep deprivation, resulting in a relative improvement in mental function. As far as the inventors are aware, there is no prior art showing that creatine could maintain or improve mental function during sleep deprivation.

The present invention enables creatine retention to be increased to a greater extent than is achieved by making simply making conventional creatine available to the body. This is achieved without the need to ingest substantial amounts of calories from simple sugars, carbohydrates and/or protein. These applications extend to individuals who do not wish to increase glycemic load including, diabetics and/or those suffering from associated disease states, people who also wish to change body composition including the loss of body fat. By maintaining plasma creatine at levels substantially the same as the optimal rate of creatine uptake by muscle, urinary creatine excretion is reduced, thereby reducing renal creatine load and increasing creatine retention. This has potential to extend the use of controlled delivery creatine to groups with impaired kidney function.

The type and amount and method of application of the delivery agent added to obtain a formulation is used to provide two important characteristics. Firstly, the resulting formulation protects the active ingredient from chemical degradation in the gastrointestinal tract. Although multiple doses of an oral formulation could be taken, it is preferable to design the dosage such that a single dose is taken up to 4 times a day. The better the active ingredient is protected from degradation the less active ingredient is needed in the original dosage, thereby reducing manufacturing costs and increasing profits. The formulation must protect at least as much of the dose as is needed to obtain a pharmacological effect, e.g. maintaining greater creatine levels in muscle and other body tissues than conventional formulations.

The second necessary characteristic of the formulation is that it does not liberate all of the active ingredient at one time but rather delivers the active ingredient gradually over time at a controlled rate, which is constant over 1 hour or more. This delivery is controlled and this is particularly important because (1) creatine has a relatively short half-life in plasma and (2) a desired level of creatine in blood, plasma, or serum must be maintained over a long period to obtain the desired effect of increasing intracellular levels. If the creatine is liberated too quickly, a large proportion will quickly enter the circulatory system and be metabolised in the liver and/or excreted in the urine. Ultimately this reduces the duration that plasma creatine is maintained at Vmax +/−30%. When this occurs the effect on creatine retention and muscle uptake will be reduced.

### EXAMPLES

A typical formulation of the invention will contain about 50% to 70% by weight of creatine and a particularly preferred formulation will comprise 60% by weight of creatine. The creatine will possess a particle size in the range 1 to 500 microns, with a preferred particle size of 1 to 20 microns. Assuming a formulation with 60% by weight of creatine with the remaining 40% being encapsulation/delivery agent and/or excipient material there are a number of possible components, which could be used to make up that 40%.

### Example 1

Creatine monohydrate 60%, with a particle size of 1 to 20 microns produced by dry bead milling, jet milling or other processes for reducing the particle size of solids, known to those experienced in the art. An enteric coat amounting to 10-15% total mass and comprised of maltodextrin (0.2 to 8%) and shellac (92% to 99.8). This is evenly coated on an inner core using fluidized bed processor with a Wurster insert, or suitable technology to achieve the same result. The inner core comprises 30% total mass and comprises a hydrophobic and hydrophilic matrix system of Carnauba wax (25-45%), Hydroxypropyl methyl cellulose (2-5%) and Sucrose (50-73%).

| | |
|---|---|
| creatine | 60% |
| organic polymer | 40% |
| TOTAL | 100% |

### Example 2

| | |
|---|---|
| creatine | 60% |
| organic polymer | 34.5% |
| Inorganics | 5.5% |
| TOTAL | 100% |

### Example 3

| | |
|---|---|
| creatine | 60% |
| organic polymer | 30%-40% |
| Inorganics | 10% or less |
| TOTAL | 100% |

### Example 4

| | |
|---|---|
| creatine | 60% |
| microcrystalline cellulose | 14% |
| cellulose acetate phthalate aqueous | 15% |
| dispersion | |
| Polyvinylpyraolidone | 3% |
| ethyl acetate | 2.5% |
| hydrous magnesium silicate (talc) | 1% |
| carboxy methyl ether | 4% |
| magnesium stearate | 0.5% |
| TOTAL | 100% |

### Example 5

| | |
|---|---|
| creatine | 60% |
| microcrystalline cellulose | 10-30% |
| cellulose acetate phthalate aqueous | 5-25% |
| dispersion | |
| Polyvinylpyraolidone | 1-5% |
| ethyl acetate | 1-5% |
| hydrous magnesium silicate (talc) | 0.5-3% |
| carboxy methyl ether | 1-5% |
| magnesium stearate | 0.5-1.5% |
| TOTAL | 100% |

### Example 6

| | |
|---|---|
| creatine | 60% |
| microcrystalline cellulose, NF | 14% |
| (Avicel PH 101) | |
| Aquacoat CPD-30 (30% solids w/w) | 15% |
| Plasdone K29/32, USP | 3% |
| Carbopol 974P, NF | 2.5% |
| Talc, USP | 1.0% |
| croscarmellose sodium, NF (Ac, di-Sol) | 4.0% |
| Magnesium Stearate, NF | 0.5% |
| TOTAL | 100% |

### Example 7

| | |
|---|---|
| creatine | 60% |
| microcrystalline cellulose, NF | 10-30% |
| (Avicel PH 101) | |
| Aquacoat CPD-30 (30% solids w/w) | 5-25% |
| Plasdone K29/32, USP | 1-5% |
| Carbopol 974P, NF | 1-5% |
| Talc, USP | 0.5-3% |
| croscarmellose sodium, NF (Ac, di-Sol) | 1-5% |
| Magnesium Stearate, NF | 0.5-1.5% |
| TOTAL | 100% |

While a preferable mode of controlled drug delivery will be oral, other modes of controlled delivery compositions according to this invention may be used. These include mucosal delivery, nasal delivery, ocular delivery, transdermal delivery, parenteral controlled delivery, vaginal delivery, rectal delivery and intrauterine delivery.

There are a number of controlled delivery drug formulations that are developed preferably for oral administration. These include, but are not limited to, osmotic pressure-controlled gastrointestinal delivery systems; hydrodynamic pressure-controlled gastrointestinal delivery systems; membrane permeation-controlled gastrointestinal delivery systems, which include microporous membrane permeation-controlled gastrointestinal delivery devices; gastric fluid-resistant intestine targeted controlled-gastrointestinal delivery devices; gel diffusion-controlled gastrointestinal delivery systems; and ion-exchange-controlled gastrointestinal delivery systems, which include cationic and anionic drugs.

Enteric coatings are applied to tablets to prevent the unwanted breakdown of drugs/nutrients in the stomach either to reduce the risk of unpleasant side effects or to maintain the stability of the drug/nutrient, which might otherwise be subject to degradation as a result of exposure to the gastric environment. Most polymers that are used for this purpose are polyacids that function by virtue or the fact that their solubility in aqueous medium is pH-dependent, and they require conditions with a pH higher then normally encountered in the stomach.

One preferable type of oral controlled delivery structure is enteric coating of a solid or liquid dosage form. Enteric coatings promote the lipoates' remaining physically incorporated in the dosage form for a specified period when exposed to gastric juice. Yet the enteric coatings are designed to disintegrate in intestinal fluid for ready absorption. Delay of the lipoates' absorption is dependent on the rate of transfer through the gastrointestinal tract, and so the rate of gastric emptying is an important factor. Some investigators have reported that a multiple-unit type dosage form, such as granules, may be superior to a single-unit type. Therefore, in a preferable embodiment, the lipoates may be contained in an enterically coated multiple-unit dosage form. In a more preferable embodiment, the lipoate dosage form is prepared by spray-coating granules of a lipoate-enteric coating agent (solid dispersion) on an inert core material. These granules can result in prolonged absorption of the drug with good bioavailability.

Typical enteric coating agents include, but are not limited to, hydroxypropylmethylcellulose phthalate, methacryclic acid-methacrylic acid ester copolymer, polyvinyl acetate-phthalate, maltodextrin, shellac, cellulose acetate phthalate and sucrose. Various enteric coating materials may be selected on the basis of testing to achieve an enteric coated dosage form designed ab initio to have a preferable combination of dissolution time, coating thicknesses and diametral crushing strength.

On occasion, the performance of an enteric coating may hinge on its permeability. With such oral drug delivery systems, the drug liberation process may be initiated by diffusion of aqueous fluids across the enteric coating. Investigations have suggested osmotic driven/rupturing affects as important delivery mechanisms from enteric-coated dosage forms.

Another type of useful oral controlled delivery structure is solid dispersion. A solid dispersion may be defined as a dispersion of one or more active ingredients in an inert carrier or matrix in the solid state prepared by the melting (fusion), solvent, or melting-solvent method. The solid dispersions may be also called solid-state dispersions. The term “coprecipitates” may also be used to refer to those preparations obtained by the solvent methods.

Solid dispersions may be used to improve the solubility's and/or dissolution rates of poorly water-soluble lipoates. The solid dispersion method was originally used to enhance the dissolution rate of slightly water-soluble medicines by dispersing the medicines into water-soluble carriers such as polyethylene glycol or polyvinylpyraolidone.

The selection of the carrier may have an influence on the dissolution characteristics of the dispersed drug because the dissolution rate of a component from a surface may be affected by other components in a multiple component mixture. For example, a water-soluble carrier may result in a fast liberation of the drug from the matrix, or a poorly soluble or insoluble carrier may lead to a slower delivery/liberation of the drug from the matrix. The solubility of the lipoates may also be increased owing to some interaction with the carriers.

Examples of carriers useful in solid dispersions according to the invention include, but are not limited to, water-soluble polymers such as polyethylene glycol, polyvinylpyraolidone, or hydroxypropylmethyl-cellulose.

Alternative carriers include phosphatidylcholine. Phosphatidylcholine is an amphoteric but water-insoluble lipid, which may improve the solubility of otherwise insoluble lipoates in an amorphous state in phosphatidylcholine solid dispersions.

Lipid Matrix Carriers (LMC) are globular structures composed of hydrophobic compounds and an amphipathic compound. These LMC's are composed primarily of lipids which provide for the controlled delivery of drugs and other biological materials including creatine. Examples of LMC technology may include the formulations disclosed in U.S. Pat. No. 4,610,868.

Other carriers include polyoxyethylene hydrogenated castor oil. Poorly water-soluble lipoates may be included in a solid dispersion system with an enteric polymer such as hydroxypropylmethylcellulose phthalate and carboxymethylethylcellulose, and a non-enteric polymer, hydroxypropylmethylcellulose. Another solid dispersion dosage form includes incorporation of the drug of interest with ethyl cellulose and stearic acid in different ratios. An alternative solid dispersion dosage form includes incorporation of the drug of interest with Alginates (e.g. sodium, calcium, magnesium) in different ratios.

There are various methods commonly known for preparing solid dispersions. These include, but are not limited to the melting method, the solvent method and the melting-solvent method.

In the melting method, the physical mixture of a drug in a water-soluble carrier is heated directly until it melts. The melted mixture is then cooled and solidified rapidly while rigorously stirred. The final solid mass is crushed, pulverized and sieved. Using this method a super saturation of a solute or drug in a system can often be obtained by quenching the melt rapidly from a high temperature. Under such conditions, the solute molecule may be arrested in solvent matrix by the instantaneous solidification process. A disadvantage is that many substances, either drugs or carriers, may decompose or evaporate during the fusion process at high temperatures. However, this evaporation problem may be avoided if the physical mixture is heated in a sealed container. Melting under a vacuum or blanket of an inert gas such as nitrogen may be employed to prevent oxidation of the drug or carrier.

The solvent method has been used in the preparation of solid solutions or mixed crystals of organic or inorganic compounds. Solvent method dispersions may be prepared by dissolving a physical mixture of two solid components in a common solvent, followed by evaporation of the solvent. The main advantage of the solvent method is that thermal decomposition of drugs or carriers may be prevented because of the low temperature required for the evaporation of organic solvents. However, some disadvantages associated with this method are the higher cost of preparation, the difficulty in completely removing liquid solvent, the possible adverse effect of its supposedly negligible amount of the solvent on the chemical stability of the drug.

Another method of producing solid dispersions is the melting-solvent method. It is possible to prepare solid dispersions by first dissolving a drug in a suitable liquid solvent and then incorporating the solution directly into a melt of polyethylene glycol, obtainable below 70 degrees, without removing the liquid solvent. The selected solvent or dissolved lipoate may be selected such that the solution is not miscible with the melt of polyethylene glycol. The polymorphic form of the lipoate may then be precipitated in the melt. Such a unique method possesses the advantages of both the melting and solvent methods.

Another controlled delivery dosage form is a complex between an ion exchange resin and the lipoates. Ion exchange resin-drug complexes have been used to formulate sustained-delivery products of acidic and basic drugs. In one preferable embodiment, a polymeric film coating is provided to the ion exchange resin-drug complex particles, making drug delivery from these particles diffusion controlled.

Injectable micro spheres are another controlled delivery dosage form. Injectable micro spheres may be prepared by non-aqueous phase separation techniques, and spray-drying techniques. Micro spheres may be prepared using polylactic acid or copoly(lactic/glycolic) acid.

Other controlled delivery technologies that may be used in the practice of this invention include SODAS, INDAS, IPDAS, MODAS, EFVAS, PRODAS, and DUREDAS. SODAS are multi particulate dosage forms utilizing controlled delivery beads. INDAS are a family of drug delivery technologies designed to increase the solubility of poorly soluble drugs. IPDAS are multi particulate tablet formations utilizing a combination of high density controlled delivery beads and an instantly degrading granulate. MODAS are controlled delivery single unit dosage forms. Each tablet consists of an inner core surrounded by a semi permeable multiparous membrane that controls the rate of drug delivery. EFVAS is an effervescent drug absorption system. PRODAS is a family of multi particulate formulations utilizing combinations of immediate delivery and controlled delivery mini-tablets. DUREDAS is a bilayer tablet formulation providing dual delivery rates within the one dosage form. Although these dosage forms are known to one of skill, certain types of these dosage forms will now be discussed in more detail.

INDAS was developed specifically to improve the solubility and absorption characteristics of poorly water-soluble drugs. Solubility and, in particular, dissolution within the fluids of the gastrointestinal tract is a key factor in determining the overall oral bioavailability of poorly water soluble drugs. By enhancing solubility, one can increase the overall bioavailability of a drug with resulting reductions in dosage. INDAS takes the form of a high-energy matrix tablet, production of which is comprised of two distinct steps: the adensosine analogue in question is converted to an amorphous form through a combination of energy, excipients, and unique processing procedures. Once converted to the desirable physical form, the resultant high-energy complex may be stabilized by an absorption process that utilizes a novel polymer cross-linked technology to prevent recrystallisation. The combination of the change in the physical state of the lipoate coupled with the solubilising characteristics of the excipients employed enhances the solubility of the lipoate. The resulting absorbed amorphous drug complex granulate may be formulated with a gel-forming erodible tablet system to promote substantially smooth and continuous absorption.

IPDAS is a multi-particulate tablet technology that may enhance the gastrointestinal tolerability of potential irritant and ulcerogenic drugs. Intestinal protection is facilitated by the multi-particulate nature of the IPDAS formulation, which promotes dispersion of an irritant lipoate throughout the gastrointestinal tract. Controlled delivery characteristics of the individual beads may avoid high concentration of drug being both liberated locally and absorbed systemically. The combination of both approaches serves to minimize the potential harm of the lipoates with resultant benefits to patients.

IPDAS is composed of numerous high-density controlled delivery beads. Each bead may be manufactured by a two-step process that involves the initial production of a micromatrix with embedded lipoates and the subsequent coating of this micromatrix with polymer solutions that form a rate limiting semi-permeable membrane in vivo. Once an IPDAS tablet is ingested, it may disintegrate and liberate the beads in the stomach. These beads may subsequently pass into the duodenum and along the gastrointestinal tract, preferably in a controlled and gradual manner, independent of the feeding state. Lipoate delivery occurs by diffusion process through the micromatrix and subsequently through the pores in the rate controlling semi-permeable membrane. The delivery rate from the IPDAS tablet may be customized to deliver a drug-specific absorption profile associated with optimised clinical benefit. Should a fast onset of activity be necessary, immediate delivery granulate may be included in the tablet. The tablet may be broken prior to administration, without substantially compromising drug delivery, if a reduced dose is required for individual titration.

MODAS is a drug delivery system that may be used to control the absorption of water-soluble lipoates. Physically MODAS is a non-disintegrating tablet formulation that manipulates drug delivery by a process of rate limiting diffusion by a semi-permeable membrane formed in vivo. The diffusion process essentially dictates the rate of presentation of drug to the gastrointestinal fluids, such that the uptake into the body is controlled. Because of the minimal use of excipients, MODAS can readily accommodate small dosage size forms. Each MODAS tablet begins as a core containing active drug plus excipients. This core is coated with a solution of insoluble polymers and soluble excipients. Once the tablet is ingested, the fluid of the gastrointestinal tract may dissolve the soluble excipients in the outer coating leaving substantially the insoluble polymer. What results is a network of tiny, narrow channels connecting fluid from the gastrointestinal tract to the inner drug core of water-soluble drug. This fluid passes through these channels, into the core, dissolving the drug, and the resultant solution of drug may diffuse out in a controlled manner. This may permit both controlled dissolution and absorption. An advantage of this system is that the drug releasing pores of the tablet are distributed over substantially the entire surface of the tablet. This facilitates uniform drug absorption reduces aggressive unidirectional drug delivery. MODAS represents a very flexible dosage form in that both the inner core and the outer semi-permeable membrane may be altered to suit the individual delivery requirements of a drug. In particular, the addition of excipients to the inner core may help to produce a microenvironment within the tablet that facilitates more predictable delivery and absorption rates. The addition of an instantly degrading outer coating may allow for development of combination products.

Additionally, PRODAS may be used to deliver lipoates according to the invention. PRODAS is a multi particulate drug delivery technology based on the production of controlled delivery mini tablets in the size range of 1.5 to 4 mm in diameter. The PRODAS technology is a hybrid of multi particulate and hydrophilic matrix tablet approaches, and may incorporate, in one dosage form, the benefits of both these drug delivery systems.

In its most basic form, PRODAS involves the direct compression of an immediate delivery granulate to produce individual mini tablets that contain lipoates. These mini tablets are subsequently incorporated into hard gels and capsules that represent the final dosage form. A more beneficial use of this technology is in the production of controlled delivery formulations. In this case, the incorporation of various polymer combinations within the granulate may delay the appearance rate of drugs from each of the individual mini tablets. These mini tablets may subsequently be coated with controlled delivery polymer solutions to provide additional delayed delivery properties. The additional coating may be necessary in the case of highly water soluble drugs or drugs that are perhaps gastroirritants where delivery can be delayed until the formulation reaches more distal regions of the gastrointestinal tract. One value of PRODAS technology lies in the inherent flexibility to formulation whereby combinations of mini tablets, each with different delivery rates, are incorporated into one dosage form. As well as potentially permitting controlled absorption over a specific period, this also may permit targeted delivery of drug to specific sites of absorption throughout the gastrointestinal tract. Combination products also may be possible using mini tablets formulated with different active ingredients.

DUREDAS is a bilayer tableting technology that may be used in the practice of the invention. DUREDAS was developed to provide for two different delivery rates, or dual delivery of a drug from one dosage form. The term bilayer refers to two separate direct compression events that take place during the tableting process. In a preferable embodiment, an immediate delivery granulate is first compressed, being followed by the addition of a controlled delivery element which is then compressed onto this initial tablet. This may give rise to the characteristic bilayer seen in the final dosage form.

The controlled delivery properties may be provided by a combination of hydrophilic polymers. In certain cases, a rapid delivery of the creatine may be desirable in order to facilitate a fast onset of therapeutic affect. Hence one layer of the tablet may be formulated as an immediate delivery granulate. By contrast, the second layer of the tablet may deliver the drug in a controlled manner, preferably through the use of hydrophilic polymers. This controlled delivery may result from a combination of diffusion and erosion through the hydrophilic polymer matrix.

A further extension of DUREDAS technology is the production of controlled delivery combination dosage forms. In this instance, two different creatine compounds may be incorporated into the bilayer tablet and the delivery of drug from each layer controlled to maximize therapeutic effect of the combination.

The creatine employed in the invention may be incorporated into any one of the aforementioned controlled delivery dosage forms, or other conventional dosage forms. The amount of creatine acid contained in each dose can be adjusted, to meet the needs of individuals and specific populations including patients.

1. A formulation for increasing creatine retention in a controlled manner in the human or animal body, comprising creatine or a derivative as the active ingredient and a delivery agent for controlling the appearance of the active ingredient in plasma whereby in use, creatine is delivered in a manner such as to increase the duration over which the plasma concentration of creatine is maintained at Vmax (+/−30%) of the muscle creatine transporters, thereby causing an increase in muscle creatine uptake.

2. The formulation of claim 1 wherein the delivery agent is selected from hydroxypropylmethylcellulose phthalate, methacryclic acid-methacrylic acid ester copolymer, polyvinyl acetate-phthalate, maltodextrin, shellac, cellulose acetate phthalate, sucrose and carnauba wax, or a combination thereof.

3. The formulation of claim 1 wherein creatine is creatine citrate, creatine pyruvate, creatine ester, or another creatine salt, chelate or conjugate.

4. The formulation of claim 1 further comprising an excipient.

5. The formulation of claim 4 wherein the excipient is selected from an orally ingested active consisting of inhibitors of liver creatine disposal selected from black pepper or black pepper extracts and other modifiers of creatine metabolism including, sodium, potassium, synephrine, 4-hydroxyisoleucine, green tea or its actives, lipoic acid, carnosine, beta-alanine, or a combination thereof.

6. The formulation of claim 1 wherein the formulation is in the form of a tablet, capsule, caplet, suspension, gel preparation, aqueous solution and solid food form, or a combination thereof.

7. The formulation of claim 1 wherein the amount of creatine ranges from 50 to 99% by weight based on total weight.

8. The formulation of claim 7 wherein the amount of creatine ranges from 50 to 70% by weight based on total weight.

9. The formulation of claim 7 wherein the amount of creatine is 60% by weight based on total weight.

10. The formulation of claim 1 wherein the amount of creatine in a single dose amount of the formulation ranges from 2 to 5 g.

11. The formulation of claim 1 wherein the formulation is suitable for oral, mucosal, nasal, ocular, transdermal, parenteral, vaginal, rectal and intrauterine delivery.

12. The formulation of claim 1 for use in increasing creatine levels in the human brain and/or spinal cord during sleep deprivation, thereby increasing cognitive function.

13. A method other than therapeutic, surgery or diagnostics, for increasing creatine retention in human or animal body by at least 10 % as compared to an identical amount of creatine given in a conventional non-controlled formulation, the method comprising administering the controlled delivery formulation of claim 1.

14. The method of claim 13 wherein a plasma concentration of creatine in humans is from 70 mg/l to 130 mg/l.

15. The method of claim 13, wherein creatine is released at a rate of 0.2 to 1.5 g per hour.

16. The method of claim 13 wherein the amount of creatine in a single dose amount of the formulation ranges from 2 to 5 g.
